(19) United States
(12) Patent Application Publication
(10) Pub. No.: US 2021/0007960 A1
(43) Pub. Date: Jan. 14, 2021

(54) SOLID HAIR CONDITIONING AGENT CONSISTING OF AT LEAST TWO COMPONENTS

(71) Applicant: Henkel AG & Co. KGaA, Duesseldorf (DE)

(72) Inventors: SOEREN SCHEELE, Pinneberg (DE); MANUELA METTE, Kleinfeld (DE); PETRA WESTPHAL, Neu Wulmstorf (DE); THOMAS SCHROEDER, Hamburg (DE)

(73) Assignee: Henkel AG & Co. KGaA, Duesseldorf (DE)

(21) Appl. No.: 16/926,065

(22) Filed: Jul. 10, 2020

(30) Foreign Application Priority Data
Jul. 10, 2019 (DE) 10 2019 210 156.9

(51) Int. Cl. A61K 8/73; A61K 8/60; A61K 8/34; A61K 8/36; A61K 8/41; A61K 8/02; A61Q 5/12

(52) U.S. Cl. CPC A61K 8/732; A61K 8/60; A61K 8/345; A61Q 5/12; A61K 8/416; A61K 8/0237; A61K 8/361

(57) ABSTRACT

A solid cosmetic composition comprises two separate Components I. and II., wherein
the at least one Component I. comprises at least one of:
starch fractions from corn, potatoes, rice, wheat and/or tapioca,
cereal flour, and/or
sugar selected from the group including glucose, sucrose, fructose, maltose, and
the at least one Component II. comprises:
from about 0.1 to 15.0% by weight of at least one cationic surfactant,
from about 10.0 to 45.0% by weight of at least one polyhydric C₂-C₆ alcohol,
from about 1.0 to 15.0% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
from about 1.0 to 20.0% by weight of at least one starch fraction, a modified starch and/or a starch derivative.

### CROSS-REFERENCE TO RELATED APPLICATION

This application claims priority to German Patent Application No. 10 2019 210 156.9, filed Jul. 10, 2019, which is incorporated herein by reference in its entirety.

### TECHNICAL FIELD

The notification describes solid cosmetic compositions based on surfactants, specific polyols, polysaccharides, fatty alcohols and/or fatty acid(s), in particular solid conditioner compositions which dissolve and foam in contact with water. The notification further describes processes for the preparation of solid cosmetic compositions and their use for conditioning mainly human hair, but also the skin of the human body.

### BACKGROUND

Surfactant-including products for conditioning primarily human hair, but also the skin of the human body, have been known for a long time and are offered on the market mainly in liquid or paste form in suitable packaging. End users take the required amount of product from the packaging during use and dispose of it after emptying. Compared to certain conditioners sold in solid form, such products offer the user the advantage of easy and quick handling, which is why they dominate the market today. However, this advantage is achieved by accepting certain disadvantages, which are discussed below. In most cases, the packaging of the described products in liquid or paste form includes non-recyclable plastic, which is a serious problem from an environmental point of view in view of the constantly growing amount of plastic waste.

Another problem is that previous products usually include higher quantities of water or water/solvent mixtures, which means that the products have a larger volume and, possibly of greater importance from a transport point of view, a relatively high weight. This is disadvantageous for several reasons. In times of increasing water scarcity, resources should be saved. An undesirable, increased transport volume associated with large-volume heavy products is also important from an environmental and cost perspective. Another interesting point is that worldwide travel activity is constantly increasing. Consumers are therefore increasingly interested in cosmetic products that are easy to transport due to their low weight and volume. This is particularly relevant with regard to air travel, as larger containers including liquids are generally excluded from being carried in an aircraft cabin, so that a passenger travelling only with hand luggage often finds himself in the situation, due to the cosmetics products that dominate the market today, of not being able to take his typical product selection with him or having to decant the corresponding products into smaller containers first, which, however, is generally accompanied by an even greater volume of packaging material.

The provision of alternative product forms with lower water content, which are contained in more environmentally friendly, for example recyclable, packaging to save space, is therefore an important goal in the formulation of improved, contemporary and sustainable cosmetic products.

Fixed conditioner compositions have been known for some time and occupy a market niche. Although they have a very low water or solvent content in general and are often packaged with little material, many people find them uncomfortable to handle because an incipient piece of conditioner is difficult to transport, often partially dissolves when placed near a shower or bathtub or next to the sink, which is also inefficient, and makes the sink or other storage location look unattractive due to conditioner residue, and because conditioner pieces have a tendency to slip out of the user's hand.

Another disadvantage with known solid conditioner formulations, especially with rather small conditioner pieces, is that it takes some time for enough of the conditioner piece to dissolve to achieve the desired amount of foam and the desired conditioning effect. On the one hand, this is usually undesirable for users because of the additional time required, and on the other hand it can be associated with higher water consumption for personal hygiene, as many users do not turn off the water flow of the shower or tap during conditioning. From this point of view, it does not make sense to market known conditioner formulations, especially in miniaturized form of a known piece of conditioner, as their dissolution is too slow as the formulations of these conditioners are not optimized for marketing in single application portions.

### BRIEF SUMMARY

In view of the problems and requirements described above, this disclosure provides formulations and manufacturing processes suitable for solid cosmetic conditioning agents which, by their nature, can be packaged in individual application portions, as well as processes for their manufacture and uses. Several measures can contribute to this suitability. Thus, the task as contemplated herein is solved by the formulation of procedures and uses described in detail below:

The present disclosure provides:

1. A solid cosmetic composition comprising two separate Components I. and II., wherein
including at least one Component I.
   a. Starch fractions from corn, potatoes, rice, wheat and/or tapioca,
   b. cereal flour and/or
   c. sugar chosen from glucose, sucrose, fructose, maltose, and
including at least one Component II.
   a. About 0.1 to 15.0% by weight of at least one cationic surfactant,
   b. About 10.0 to 45.0% by weight of at least one polyhydric C₂-C₆ alcohol
   c. About 1.0 to 15.0% by weight of at least one saturated or unsaturated, branched or unbranched C₈-C₃₀ alcohol and/or a saturated or unsaturated, branched or unbranched C₅-C₃₀ carboxylic acid and/or a salt of a saturated or unsaturated, branched or unbranched C₈-C₃₀ carboxylic acid, and
   d. About 1.0 to 20.0% by weight of at least one starch fraction, a modified starch
and/or a starch derivative,

where the quantities stated refer to the total weight of Component II.

2. A solid cosmetic composition according to point 1, including in Component I.—based on its total weight—about 75 to 100% by weight of one or more ingredient(s) from groups a. to c.
3. A solid cosmetic composition according to one of points 1 or 2, including in Component I.—based on its total weight—about 75 to 100% by weight of an ingredient from Group a.
4. A solid cosmetic composition according to one of points 1 to 3, including in Component I.—based on its total weight—about 75 to 100% by weight potato starch.
5. A solid cosmetic composition according to any of the preceding points, including in Component I.—based on its total weight—
a. About 0 to 10% by weight of native vegetable oils and/or
b. About 0 to 15% by weight of water.
6. A solid cosmetic composition according to any of the preceding points, including in Component II.—based on the total weight of Component II.—about 0.1 to 15% by weight, typically about 0.25 to 10% by weight and particularly typically about 0.5 to 7.5% by weight of at least one cationic surfactant a).
7. A solid cosmetic composition according to any of the preceding points, including as cationic surfactant a) in Component II. at least one compound from the following group of
i. Alkyl quats,
ii. Esterquats,
iii. quaternary imidazolines,
iv. Amidoamines and/or cationized Amidoamines and
v. Mixtures of these.
8. A solid cosmetic composition according to one of the preceding points 6 or 7, including in Component II. one or more surfactants from groups i. to v. in amounts of about 0.1 to 15% by weight, typically about 0.25 to 10% by weight and particularly typically about 0.5 to 7.5% by weight (based on the total weight of Component II.)
9. A solid cosmetic composition according to one of the preceding points 7 or 8, including in Component II. at least one cationic surfactant from Group i.
10. A solid cosmetic composition according to one of the preceding points 7 to 9, including in Component II C₈-C₃₀ alkyl tri-C₁-C₄-alkyl ammonium salts, typically C₈-C₂₄ alkyl trimethylammonium salts, particularly typically lauryl trimethylammonium salts, cetyl trimethylammonium salts, stearyl trimethylammonium salts, behen trimethylammonium salts and/or mixtures thereof.
11. A solid cosmetic composition according to one of the preceding points 7 or 10, including in Component II. at least one cationic surfactant a) known under the INCI designation “Cetrimonium” and/or “Behentrimonium”.
12. A solid cosmetic composition according to one of points 7 or 8, including in Component II. Cetrimonium chloride.
13. A solid cosmetic composition according to any of the preceding points, including in Component II.—based on the total weight of Component II.—about 10.0 to about 45% by weight, typically about 15.0 to about 40.0% by weight, particularly typically about 20.0 to about 35.0% by weight of at least one polyhydric C₂-C₆ alcohol b).
14. A solid cosmetic composition according to any of the preceding points, including as polyhydric alcohol b) in Component II. 1.2-propylene glycol, 1.3-butylene glycol, dipropylene glycol, glycerol and/or diglycerol
15. A solid cosmetic composition according to one of points 13 or 14, including glycerol.
16. A solid cosmetic composition according to any of the preceding points, including in Component II.—based on the total weight of Component II.—about 1 to 15% by weight, typically about 2 to 12% by weight, particularly typically about 8 to 12% by weight of Component c).
17. A solid cosmetic composition according to any of the preceding points, including as component c) in Component II. saturated or unsaturated, branched or unbranched C₈-C₃₀ carboxylic acids and/or saturated or unsaturated, branched or unbranched C₈-C₃₀ alcohols, typically C₁₀-C₂₂ carboxylic acids and/or C₁₀-C₂₂ alcohols and in particular coco acids, Lauric acid, myristic acid, palmitic acid, stearic acid, behenic acid, oleic acid and mixtures thereof and/or coconut alcohols, lauryl alcohol, myristyl alcohol, cetyl alcohol, stearyl alcohol, behenyl alcohol, oleyl alcohol and mixtures thereof.
18. A solid cosmetic composition according to one of the preceding points 16 or 17, comprising
Cetyl alcohol,
Stearyl alcohol,
Palmitic acid,
Stearic acid and/or
Mixtures of these.
   19. A solid cosmetic composition according to any of the preceding points, including in Component II.—based on the total weight of Component II.—about 1 to 20% by weight, typically about 5 to 15% by weight, particularly typically about 8 to 12% by weight of Component d)
   20. A solid cosmetic composition according to any of the preceding points, including as Polysaccharides) in Component II.
   vi. Starch fractions from corn, potatoes, rice, wheat and/or tapioca and/or
   vii. Starch fractions from corn, potatoes, rice, wheat and/or tapioca and/or
   viii. Derivatives of starches such as amylose, amylopectin, dextrins.
   21. A solid cosmetic composition according to point 20, including as polysaccharide d)i. starch fractions from corn.
   22. A solid cosmetic composition according to point 20, including as polysaccharide d)ii. compounds known under the INCI designation Hydroxypropyl Starch Phosphates.
   23. A solid cosmetic composition according to point 20, including as polysaccharide d)iii Maltodextrin.
   24. A solid cosmetic composition according to point 20, including polysaccharides d) from groups i, ii and iii, typically starch fractions from maize, compounds known under the INCI designation Hydroxypropyl Starch Phosphates and maltodextrin.
   25. A solid cosmetic composition according to any of the preceding points, including in Component II.—based on the total weight of Component II.—about 0.01 to 10% by weight, typically about 0.02 to 8% by weight, more typically about 0.05 to 6% by weight, more typically about 0.1 to 5% by weight of at least one oil, fat and/or wax component, typically a naturally occurring oil, fat or wax.
   26. A solid cosmetic composition according to point 25, including vegetable oils and/or vegetable butters
   27. A solid cosmetic composition according to one of the preceding points 25 or 26, including Shea butter (INCI designation: Butyrospermum Parkii (Shea) Butter).
   28. A solid cosmetic composition according to one of the preceding points 25 or 26, including apricot kernel oil, argan oil, jojoba oil, marula oil, almond oil, olive oil, coconut oil and/or sunflower oil.
   29. A solid cosmetic composition according to any of the preceding points, including in Component II.—based on the total weight of Component II.—Water in an amount of up to about 50% by weight, typically about 47.5% by weight and in particular about 45% by weight.
   30. A solid cosmetic composition according to any of the preceding points for the care of human hair, in particular for use after hair cleansing as a leave-on or rinse-off composition
   31. A solid cosmetic composition according to any of points 1 to 30, wherein
Component I. was coated with Component II., or
Component I. has been impregnated, sprayed or coated with Component II., or
Component II. has been rolled onto Component I., or
Component I. with Component II. was printed.
   32. A solid cosmetic composition according to one of the points 1 to 30, where Component II. is located as a separate layer between two Components I. (sandwich arrangement).
   33. A solid cosmetic composition according to any one of points 1 to 30, comprising an additional layer III including a hair cleansing agent.
   34. A solid cosmetic composition according to any of points 1 to 30, where Component II. is completely enveloped by Component I.
   35. A solid cosmetic composition according to any one of points 31 to 34, wherein Component II. is in the form of a porous body which has a density in the range about 0.2 g/cm³ to about 1.2 g/cm³ and which transforms into an emulsion in contact with water.
   36. A cosmetic method for hair care in which a solid cosmetic composition is moistened, soaked and/or emulsified with water according to one of the preceding points, rubbed between the hands and distributed on the hair, and after an exposure time of about 5 seconds to about 5 minutes, optionally rinsed again with water.
   37. A method of applying a cosmetic composition according to one of points 31 to 35, wherein
   a) the solid composition is first mixed with water and then applied to the hair, or
   b) the solid composition is applied directly to the hair and worked in.
   38. The use of a cosmetic composition according to any of the preceding points for hair care.

In one embodiment, the disclosure provides a solid cosmetic composition comprising two separate Components I. and II., wherein
the at least one Component I. comprises at least one of:

a. starch fractions from corn, potatoes, rice, wheat and/or tapioca,

b. cereal flour, and/or

c. sugar selected from the group including glucose, sucrose, fructose, maltose, and
the at least one Component II. comprises:

a. from about 0.1 to 15.0% by weight of at least one cationic surfactant,

b. from about 10.0 to 45.0% by weight of at least one polyhydric C₂-C₆ alcohol,

c. from about 1.0 to 15.0% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and

d. from about 1.0 to 20.0% by weight of at least one starch fraction, a modified starch and/or a starch derivative,
where the quantities stated refer to the total weight of Component II.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

A first subject of this application is a solid cosmetic composition comprising two separate Components I. and II.
including at least one Component I.

a. Starch fractions from corn, potatoes, rice, wheat and/or tapioca,

b. cereal flour and/or

c. sugar chosen from glucose, sucrose, fructose, maltose, and
including at least one Component II.

a. about 0.1 to 15.0% by weight of at least one cationic surfactant,

b. about 10.0 to 45.0% by weight of at least one polyhydric C₂-C₆ alcohol

c. about 1.0 to 15.0% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and

d. about 1.0 to 20.0% by weight of at least one starch fraction, a modified starch and/or a starch derivative,

where the quantities stated refer to the total weight of Component II.

The compositions as contemplated herein are solid at about 25° C. Solid compositions within the meaning of the present application are three-dimensional, dimensionally stable entities which are not liquid or gaseous, that is to say, which retain their external shape even without a surrounding vessel. However, the term “solid” does not imply anything about density or elasticity or other physical properties, so that jellies, brawn, butter etc. can also be solid as contemplated herein as long as they are dimensionally stable at about 25° C.

Component I serves here, for example, as a coating, matrix and/or base (in the form of a thin flake and/or wafer) for Component II. This creates more freedom in the formulation of Component II, as its strength and surface properties are less restricted. The ingredients listed for Component I. come from natural raw materials, are therefore sustainably usable, harmless to humans and the environment and are well suited for this application due to their structure-giving properties.

Component I. may contain cosmetic active ingredients for skin and/or hair care. However, this is not absolutely necessary as typically the active substances required for skin and/or hair care are contained in Component II. of the solid compositions as contemplated herein.

Component II represents the primarily cosmetically effective composition.

A formulation as just described offers the right properties for single application portions, especially with regard to its dissolution behaviour during use. The high concentrations of the active substances in such a composition are associated with the advantages that few resources are consumed during production and transport and that the products, even after they have reached the hands of a consumer via the trade, can be easily transported without great effort or restrictions, whether to the gym or on a flight.

### Description of the Component I.:

The present disclosure further relates to a solid cosmetic composition as described above, including in Component I.—based on its total weight—about 75 to 100% by weight of one or more ingredient(s) from groups a. to c., typically about 75 to 100% by weight of an ingredient from group a. and in particular about 75 to 100% by weight of potato starch.

The mentioned polysaccharides (I.a.), in particular potato starch, have proved to be well suited as stabilising agents in the context as contemplated herein claimed here. Their use makes it possible to provide ready-made consumer products that retain their properties and appearance over a long period of time and under various environmental conditions. These advantages are particularly pronounced when implementing the concentration ranges mentioned.

The present disclosure further relates to a solid cosmetic composition as described above, including in Component I.—based on its total weight—
about 0 to 10% by weight of native vegetable oils and/or
about 0 to 15% by weight of water.

The solid cosmetic compositions as described above typically contain in Component I.—based on the total weight of Component I. —
about 0 to 8, more typically about 0 to 6% by weight, more typically about 0 to 4% by weight and more typically about 0 to 2% by weight of native vegetable oils and/or
about 0 to 15% by weight, more typically about 0 to 14% by weight, more typically about 0 to 12.5% by weight and more typically about 0 to 10% by weight of water

Specific suitable oils will be discussed later. Naturally occurring raw materials have the advantage that they grow again and can therefore be used sustainably. This aspect is also becoming increasingly important to many users. In addition, some vegetable oils or butters, especially if they have been carefully extracted at low temperatures, are extremely potent care products for skin and hair, as they also contain a large number of certain secondary ingredients such as vitamins. A certain water content may be advantageous to adjust the properties of the finished solid cosmetic composition and/or to facilitate its preparation.

### Description of Component II.:

Cationic surfactants carry a positive charge in their hydrophilic part. This positive charge causes the surfactant molecules to attach themselves to the negatively charged skin and hair surface. In this way they neutralize the charge, prevent the hair from flying, have a smoothing effect, increase hair shine and improve wet comb-ability. They are primarily used in conditioners, hair conditioners and hair treatments, rarely in shampoos. In addition, cationic surfactants have a co-conserving effect in cosmetic products due to their bactericidal action, i.e. a bacteria-inhibiting effect.

In principle, all cationic surface-active substances suitable for use on the human body are suitable as cationic surfactants in compositions as contemplated herein. These are exemplified by at least one water-solubilizing, cationic group, such as a quaternary ammonium group, or by at least one water-solubilizing, cationizable group, such as an amine group and furthermore at least one lipophilic alkyl group with about 6 to 30 C atoms, or also by at least one imidazole group or at least one imidazylalkyl group.

In general, cationic surfactants are divided into groups according to their structural characteristics. Particularly suitable for use in the compositions as contemplated herein are cationic surfactants a) from at least one of the groups of alkylquats, esterquats, quaternary imidazolines, amidoamines and/or cationized amidoamines.

The present disclosure thus also relates to a solid cosmetic composition as described above, including as cationic surfactant a) in Component II. at least one compound from the following group of

i. Alkylquats,
ii. Esterquats,
iii. quaternary imidazolines,
iv. Amidoamines and/or cationized Amidoamines and
v. Mixtures of these.

These specifically named cationic surfactant salts have shown in Component II. of the compositions as contemplated herein a conditioning effect which is perceived as particularly pleasant.

Especially typical compositions as contemplated herein contain in Component II. as cationic surfactant a)
quaternary ammonium compounds (alkylquats) with at least one C₈-C₂₄ alkyl radical
Esterquats,
Amidoamines with at least one C₈-C₂₄ alkyl group
and mixtures thereof.

Quaternary ammonium compounds with at least one C₈-C₂₄ alkyl radical are particularly typical ammonium halides, especially chlorides, and ammonium alkyl sulphates, such as methosulphates or ethosulphates, such as C₈-C₂₄ alkyl trimethylammonium chlorides, C₈-C₂₄ dialkyldimethylammonium chlorides and C₈-C₂₄ trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known under the INCI designations Quatemium-27, Quaternium-83, Quaternium-87 and Quaternium-91. The alkyl chains of the surfactants mentioned above typically have 8 to 24 carbon atoms.

Esterquats are cationic surfactants which contain both at least one ester function and at least one quaternary ammonium group as a structural element and furthermore at least one C₈-C₂₄ alkyl radical or C₈-C₂₄ acyl radical. Typical esterquats are quaternized ester salts of fatty acids with triethanolamine, quaternized ester salts of fatty acids with diethanolalkylamines and quaternized ester salts of fatty acids with 1.2-dihydroxypropyl dialkylamines. Such products are sold under the trademarks Stepantex®, Dehyquart® and Armocare®. N,N-Bis(2-palmitoyloxyethyl)dimethylammonium chloride, distearoylethyl dimonium methosulfates, distearoylethyl hydroxyethylmonium methosulfates and bis-(isostearoyl/oleoyl isopropyl) dimonium methosulfates are typical examples of such esterquats.

Bis-(Isostearoyl/oleoyl isopropyl) dimonium methosulphate is a particularly typical esterquat.

The alkylamidoamines are usually produced by amidation of natural or synthetic C₈-C₂₄ fatty acids and fatty acid sections with di-(C₁-C₃)alkylaminoamines. Compounds from this substance group which are particularly suitable as contemplated herein are for example the compounds known under the INCI designations stearamidopropyl dimethylamine, behenamidopropyl dimethylamine and/or brassicamidopropyl dimethylamine. Stearamidopropyl dimethylamine is particularly typical.

The present disclosure also relates in a typical embodiment to a solid cosmetic composition as described above, including in Component II. at least one cationic surfactant a) from group i., typically C₈-C₃₀ alkyl-tri-C₁-C₄ alkylammonium salts and in particular cationic surfactant salts known under the INCI designation “cetrimonium” and/or “behentrimonium”.

Cetrimonium chloride is particularly typical.

The present disclosure also relates in a further typical embodiment to a solid cosmetic composition as described above, including in Component II. at least one cationic surfactant a) from group ii., typically quaternized ester salts of fatty acids with diethanolalkylamines and in particular cationic surfactant salts (esterquats ii.) known under the INCI designation “Bis-(isostearoyl/oleoyl isopropyl) dimonium”.

Compositions typical as contemplated herein contain in Component II. at least one cationic surfactant a) in a total amount of about 0.1 to 15% by weight, typically about 0.25 to 10% by weight, particularly typically about 0.5 to 7.5% by weight, based on the weight of Component II.

In addition to conditioning agents, the compositions as contemplated herein can also be cleaning agents. Typical cleaning agents as contemplated herein can typically be
at least one cationic surfactant, typically in a total amount of about 0.1 to 2% by weight, more typically about 0.2 to 1% by weight and more typically about 0.3 to 0.5% by weight, each based on the weight of the composition, and
contain at least one further surfactant chosen from anionic, amphoteric, zwitterionic and/or non-ionic surfactants, typically in a total amount of about 1 to 40% by weight, more typically about 2 to 35% by weight and particularly typically about 3 to 30% by weight, each based on the weight of the composition.

Compositions typical as contemplated herein also contain in Component II. at least one polyhydric C₂-C₆-alcohol in a total amount of about 10.0 to 45.0% by weight, typically about 15.0 to 40.0% by weight, particularly typically about 20.0 to 35.0% by weight, each based on the weight of Component II.

The present disclosure also concerns a solid cosmetic composition as described above, including as polyhydric alcohol b) in Component II.
Alditols such as mannitol, isomalt, lactitol, sorbitol and xylitol, threat, erythritol and arabitol
Glycols such as 1.2-propylene glycol, 1.3-butylene glycol, 1.6-hexanediol,
Triols or polyols such as dipropylene glycol, glycerol and/or diglycerol.

Typical alcohols b) are 1.2-propylene glycol, 1.3-butylene glycol, dipropylene glycol, glycerol and/or diglycerol. In particular, typically including solid compositions as contemplated herein in Component II. Glycerol in the above-mentioned quantities.

These polyhydric alcohols are well tolerated by the skin and as solvents they ensure that the solid cosmetic compositions available with them are not too solid or too difficult or slow to dissolve.

The present disclosure also relates to a solid cosmetic composition as described above, including in Component II as Component c) saturated or unsaturated, branched or unbranched C₈-C₃₀ carboxylic acids and/or saturated or unsaturated, branched or unbranched C₈-C₃₀ alcohols, typically C₁₀-C₂₂ carboxylic acids and/or C₁₀-C₂₂ alcohols and in particular coco acids, Lauric acid, myristic acid, palmitic acid, stearic acid, behenic acid, oleic acid and mixtures thereof and/or coconut alcohols, lauryl alcohol, myristyl alcohol, cetyl alcohol, stearyl alcohol, behenyl alcohol, oleyl alcohol and mixtures thereof.

In particular, solid cosmetic compositions contained in Component II. Cetyl alcohol, stearyl alcohol, palmitic acid, stearic acid and/or mixtures thereof in the proportions or concentrations mentioned.

These compounds have proved to be particularly suitable structure-giving ingredients for the purposes as contemplated herein. They can be used to formulate cosmetic compositions of sufficient strength that do not melt too low. They are contained in Component II. of the compositions as contemplated herein typically at about 1 to 15% by weight, typically about 2 to 12% by weight, particularly typically about 8 to 12% by weight, each based on the weight of Component II.

Polysaccharides suitable for the present disclosure d) are composed of more than ten monosaccharide units. Typical polysaccharides are the starches composed of u-D-glucose units and starch degradation product s such as amylose, amylopectin and dextrins. As contemplated herein, chemically and/or thermally modified starches, e.g. hydroxypropyl starch phosphate, dihydroxypropyl distarch phosphate or the commercial products Dry Flo® are also particularly advantageous. Dextranes and their derivatives, e.g. dextran sulphate, are also typical. Also typical are non-ionic cellulose derivatives, such as methyl cellulose, hydroxypropyl cellulose, hydroxypropyl methyl cellulose or hydroxyethyl cellulose, and cationic cellulose derivatives, e.g. the commercial products Celquat® and Polymer JR®, and typically Celquat® H 100, Celquat® L 200 and Polymer JR 400 (polyquatemium-10) and polyquatemium-24. Other typical examples are polysaccharides from fucose units, such as the commercial product Fucogel®.

In Component II. of the compositions as contemplated herein, the polysaccharides d) are typically contained in amounts of about 1 to 20% by weight, typically about 5 to 15% by weight and particularly typically about 8 to 12% by weight, each based on the weight of Component II.

The present disclosure also relates to a solid cosmetic composition as described above, including as polysaccharide d) in Component II.

i. Starch fractions from corn, potatoes, rice, wheat and/or tapioca and/or
ii. Starch fractions from corn, potatoes, rice, wheat and/or tapioca and/or
iii. Derivatives of starches such as amylose, amylopectin, dextrins,

Particularly typical is a solid cosmetic composition, as described above, including as polysaccharide d)i. in Component II Starch fractions from corn.

Also particularly typical is a solid cosmetic composition, as described above, including as polysaccharide d)ii. in Component II. a compound known under the INCI designation Hydroxypropyl Starch Phosphates.

Also particularly typical is a solid cosmetic composition, as described above, including as polysaccharide d)iii. in Component II. Maltodextrin.

Especially typical is a solid cosmetic composition, as described above, including in Component II. Polysaccharides (d) from groups i, ii and iii, typically starch fractions from maize, compounds known under the INCI designation Hydroxypropyl Starch Phosphates and maltodextrin.

These polysaccharides have proved to be well suited as stabilising agents in the context as contemplated herein claimed here. Their use makes it possible to provide ready-made consumer products that retain their properties and appearance over a long period of time and under various environmental conditions.

The present disclosure also relates to a solid cosmetic composition as described above, including in Component II—based on the total weight of Component II.—about 0.01 to 10.00% by weight of at least one oil, fat and/or wax component, typically a naturally occurring oil, fat or wax.

These are caring substances that help to keep both the skin and hair structure healthy. The defined concentration range makes it possible to use this care effect but at the same time to exclude noticeable greasiness after application of an appropriate composition. Naturally occurring raw materials have the advantage that they grow again and can therefore be used sustainably. This aspect is also becoming increasingly important to many users.

The present disclosure also concerns a solid cosmetic composition as described above, including in Component II. vegetable oils and/or vegetable butters.

As already mentioned, naturally occurring raw materials have the advantage that they grow again and can therefore be used sustainably. This aspect is also becoming increasingly important to many users. In addition, some vegetable oils or butters, especially if they have been carefully extracted at low temperatures, are extremely potent care products for skin and hair, as they also contain a large number of certain secondary ingredients such as vitamins.

It has been found that vegetable butters with a melting range of about 20° C. to 35° C. are particularly suitable for incorporation into cosmetic compositions as contemplated herein.

Accordingly, vegetable butters with a melting point in the range from about 20° C. to 35° C., such as Shea butter (INCI designation), are particularly typical: Butyrospermum Parkii (Shea) Butter), Mango Butter (INCI designation: Mangifera Indica (Mango) Seed Butter), Murumuru Butter (INCI designation: Astrocaryum Murumuru Seed Butter), cocoa butter (INCI designation: Theobroma Cacao (Cocoa) Seed Butter) and/or Cupuacu Butter (INCI designation: Theobroma Grandiflorum Seed Butter).

Cupuacu butter (INCI designation) is particularly typical: Theobroma Grandiflorum Seed Butter) and/or Shea Butter (INCI designation: Butyrospermum Parkii (Shea) Butter) and especially typical is Shea Butter (INCI designation: Butyrospermum Parkii (Shea) Butter).

The at least one vegetable butter (typically Cupuacu butter and/or Shea butter; especially Shea butter) is used in Component II. of the cosmetic compositions as contemplated herein typically in a proportion by weight of about 0.01 to 10.00% by weight, more typically of about 0.05 to 5% by weight, particularly typically of about 0.10 to 1% by weight of the total weight of Component II.

Oils suitable as contemplated herein are typically perfume oils and/or vegetable triglyceride oils, such as coconut oil, (sweet) almond oil, walnut oil, peach kernel oil, apricot kernel oil, avocado oil, tea tree oil, soy bean oil, cotton seed oil, sesame oil, sunflower oil, tsubaki oil, evening primrose oil, rice bran oil, palm oil, Palm kernel oil, mango kernel oil, cranberry oil, sea buckthorn oil, meadow foam herb oil, thistle oil, macadamia nut oil, grape seed oil, amaranth seed oil, argan oil, bamboo oil, olive oil, Wheat germ oil, pumpkin seed oil, mallow oil, hazelnut oil, safflower oil, corn oil, olive oil, rapeseed oil, canola oil, sasanqua oil, jojoba oil, rambutan oil, marula oil and/or quinoa oil.

Particularly typical are apricot kernel oil, argan oil, jojoba oil, marula oil, macadamia nut oil, pumpkin seed oil, amaranth seed oil, quinoa oil, soy bean oil, cotton seed oil, sunflower oil, palm oil, palm kernel oil, linseed oil, almond oil, corn oil, olive oil, rapeseed oil, sesame oil, soy bean oil, thistle oil, wheat germ oil, peach kernel oil, cranberry oil, sea buckthorn oil and/or coconut oil.

Especially typical are apricot kernel oil, argan oil, jojoba oil, marula oil, almond oil, olive oil, coconut oil and/or sunflower oil.

The oil(s) can be used in Component II. of the compositions as contemplated herein typically in a proportion by weight of about 0.01 to 10%, more typically about 0.05 to 7%, particularly typically about 0.10 to 5%, of the total weight of Component II.

In another typical embodiment, the present disclosure relates to solid compositions, which in Component II. (based on the total weight of Component II.) Water in an amount of up to about 50% by weight, typically about 47.5% by weight and in particular about 45% by weight.

In addition to the ingredients described above, the cosmetic compositions as contemplated herein may contain at least one active ingredient advantageously chosen from plant extracts, humectants, protein hydrolysates, perfumes, UV filters, structurants such as maleic acid, dyes for colouring the composition, Active ingredients such as bisabolol and/or allantoin, antioxidants, preservatives such as sodium benzoate or salicylic acid, additional viscosity regulators such as salts (NaCl) or polymers, and pH adjusters such as α- and β-hydroxycarboxylic acids such as citric acid, lactic acid, malic acid, glycolic acid, and/or bases such as alkanolamines and/or sodium hydroxide).

Suitable plant extracts are extracts that can be produced from all parts of a plant. Usually these extracts are produced by extraction of the whole plant. However, in some cases it may be preferable to produce the extracts exclusively from flowers and/or leaves of the plant.

Especially suitable are extracts from Paeonia Lactiflora, Rosa Darnascena Flower, Malus Domestica Fruit, Argania Spinosa Shell Powder, Larninaria Saccharina, Cannabis Sativa, Green Tea, Oak bark, Nettle, Hamanelis, Hops, Chamomile, Burdock root, Horsetail, Hawthorn, Lime blossom, Litchi, Almond, Aloe Vera, Spruce needle, Horse chestnut, Sandalwood, juniper, coconut, mango, apricot, lime, wheat, kiwi, melon, orange, grapefruit, sage, rosemary, birch, mallow, cuckooflower, thyme, yarrow, Thyme, lemon balm, cowslip, marshmallow, ginseng, ginger root, Echinacea purpurea, Olea europea, Boerhavia diffusa roots, Foeniculum vulgaris and Apirn graveolens.

The extracts of Paeonia Lactiflora, Rosa Damascena Flower, Malus Domestica Fruit, Argania Spinosa Shell Powder, Laminaria Saccharina, Cannabis Sativa, Green Tea, Nettle, Hamamelis, Chamomile, Aloe Vera, Ginseng, Echinacea purpurea, Olea europea and/or Boerhavia Diffusa roots are particularly typical for use in the compositions as contemplated herein.

Water, alcohols and mixtures thereof may be used as extraction agents for the preparation of the above plant extracts. Among the alcohols, lower alcohols such as ethanol and isopropanol, but especially polyhydric alcohols such as ethylene glycol and propylene glycol, both as the sole extracting agent and mixed with water, are typical. Plant extracts based on water/propylene glycol in a ratio of about 1:10 to 10:1 have proven to be particularly suitable.

The plant extracts can be used both in pure and diluted form. If they are used in diluted form, they usually contain approx. 2-80% by weight of active substance and the extraction agent or mixture of extraction agents used in their extraction as solvent. The plant extracts can be used in Component 11. as contemplated herein (based on the total weight of Component I.) typically in an amount of about 0.01 to 10% by weight, more typically of about 0.05 to 7.5% by weight and in particular of about 0.1 to 5% by weight.

The present disclosure further relates to a solid cosmetic composition as described above, for the care of human hair, in particular for use after hair cleansing as a leave-on or rinse-off composition.

The terms ‘leave-on’ and ‘rinse-off’ mean that the composition is left in the hair either for a relatively short period of time, such as less than a minute, or for a few minutes or an hour, until it is rinsed out, or that the composition remains in the hair until the next wash, which may be a few days. Both have certain advantages. With a composition that remains on the hair for a long time, the full care potential of all ingredients can be used to a certain extent, whereas a composition that is to be rinsed out again in a short time can also contain ingredients that have a good care effect but whose longer retention in the hair would be unpleasant.

Typical in terms of the present disclosure are rinse-off compositions.

The present disclosure further relates to a solid cosmetic composition as described above, where
Component I. was coated with Component II., or
Component I. has been impregnated, sprayed or coated with Component II., or
Component II. has been rolled onto Component I., or
Component I. with Component II. was printed.

This offers the advantage of allowing the effects of the two different components to occur at different times. In addition, in this way it is possible to use a component on the inside which, on its own, would appear unattractive, for example because it would be too soft and attract small dust particles and the like. It may be attractive to be able to use such a component nevertheless, as this increases the degrees of freedom for the formulation. Apart from that, these cosmetic compositions appeal to the playful side of the user and can be enriching due to their novel properties.

The present disclosure further relates to a solid cosmetic composition as described above, whereby Component II. is located as a separate layer between two components I. (sandwich arrangement).

This design offers the advantages just described to a particular extent, while at the same time being easy to manufacture.

The present disclosure further relates to a solid cosmetic composition as described above, comprising an additional layer III including a (hair) cleansing agent.

A (hair) cleaning agent in the sense of the present disclosure is typically understood to be a solid cleaning composition which—based on the total weight of the layer III.—is typical
a) about 10 to 50% by weight of at least one anionic, amphoteric, zwitterionic and/or non-ionic surfactant,
b) about 10 to 60% by weight of at least one polyhydric C₂-C₆ alcohol,
about 0.1 to 10.% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
d) about 0.1 to 20% by weight of at least one polysaccharide chosen from
   Starch fractions and/or derivatives of starches and/or
   Cellulose and/or cellulose derivatives

This gives the composition additional cleaning properties. In addition to a pleasant skin or hair feeling caused by the conditioning ingredients, these have a cleansing effect and thus offer added value. In the context as contemplated herein, this is possible without significantly affecting the conditioning performance. The aforementioned three-layer structure is particularly suitable for this purpose.

The present disclosure further relates to a solid cosmetic composition as described above, Component II. being completely enveloped by Component I.

Such a design achieves the advantages previously described in connection with the spatial arrangement of the components to a particular extent.

The present disclosure further relates to a solid cosmetic composition as previously described, wherein Component II. is in the form of a porous body which has a density in the range of about 0.2 g/cm³ to 1.2 g/cm³ and which transforms into an emulsion in contact with water.

A porous body feels interesting, which enriches the user sensorially, and also dissolves well and quickly due to the large surface, which can save time but also water.

The present disclosure further relates to a cosmetic method for hair care in which a solid cosmetic composition is moistened, soaked and/or emulsified with water as described above, rubbed between the hands and distributed on the hair, and after an exposure time of about 5 seconds to 5 minutes is optionally rinsed out again with water.

The present disclosure also relates to a method of applying the solid cosmetic composition as contemplated herein, in which the solid composition is either first mixed with water and then applied to the hair, or in which the solid composition is applied directly to the wet hair.

These procedures make the advantages described above tangible for a user and thus represent an enrichment not only for personal hygiene and care but also a sensory enrichment.

The present disclosure further relates to a use of a cosmetic composition as described above for hair care.

This use makes the advantages described above tangible for a user and thus represents an enrichment not only for personal hygiene and care but also a sensory enrichment.

As can be seen from the previously described designs and their advantages, the process and packaging aspects are important for the present disclosure. They are discussed in more detail below.

In a manufacturing process as contemplated herein (for Component II.), for example, all ingredients are placed in a heat able container, such as, on a laboratory scale, in a suitable vessel in a water bath or on a heating plate, on a production scale rather in a closed and pressurizable vessel, and are mixed and heated, in the recipes as contemplated herein, for example at about 75° C. until all ingredients are sufficiently mixed. In such a process, different temperature steps can also be run. For example, components that can be homogeneously mixed even at a relatively low temperature can be mixed first. This can happen at about 40° C. to 50° C. It can also be advantageous to mix in certain ingredients at higher temperatures, for example at about 85° C. to 90° C. For this purpose, a process as contemplated herein may comprise one or more steps in this temperature range. Afterwards, one or more steps can be carried out at a lower temperature again, in which further components are mixed in. Typically, the compositions as contemplated herein solidify at about 65° C., so that certain process steps, such as mixing and extruding the finished mixtures, are not reasonably possible below such a temperature level.

A mixture resulting from a process described above may also contain a gas, including gas mixtures such as air, N₂, N₂O and/or CO₂. This can be done in a boiler, for example, at about 200-4000 kPa or by adding air, for example, using a high-speed mixer or similar equipment. A mixture thus obtained can then be discharged via an extruder. The mixture expands if it was too pre-pressurized, as discussed here, and solidifies at a normal room temperature of about 18° C. to 25° C., for example, additionally favoured by cooling, which is accompanied by the expansion of the contained gas. Otherwise, if it was previously mixed under ambient pressure, the mixture will only cool down and solidify by assuming the ambient temperature or by additionally provided cooling.

The introduction of a gas or gas mixture into the compositions as contemplated herein (Component II.) is accompanied by various advantages. As explained at the beginning, good dissolving properties are important for solid cosmetic compositions, in particular for compositions which are also intended to be used for single application portions. In addition to the composition, the dissolution behaviour can be influenced by the method of production and packaging. For example, by introducing a gas or gas mixture, the surface area where contact with water can take place can be increased, resulting in faster dissolution, and the extremely fine bubble structure already established in the solid composition means that the foam resulting from dissolution is particularly fine and creamy, which is perceived as pleasant.

It is also important to note that cosmetic products have a lot to do with feeling, fun and emotions. Many people relax during personal hygiene and enjoy the pleasant feeling of doing something good for themselves. Especially since many people find their everyday life more and more demanding or stressful, small pleasures and playfulness are an important point at which stress can be released from a person and satisfaction can be created. Solid cosmetic preparations with incorporated gas phase, i.e. to a certain extent solid foams, feel different from conventional products, which is perceived as interesting and pleasant.

The extrusion processes described above can also be used to produce interesting shapes reminiscent of injection-moulded biscuits. Thanks to specially shaped dies on the extrusion die, a variety of shapes can be realized, for example a heart or clover shape. An extruded strand thus obtained can then be cut into pieces or slices, providing emotionally appealing small portions of the solid cosmetic compositions as contemplated herein. Similarly, it is possible to roll out an extruded strand or other form of a composition as contemplated herein and then, by punching or cutting, produce pieces that are similar to cookie cutters in terms of shaping properties.

There are further possibilities to create exciting, emotionally appealing products through a special implementation of a manufacturing process. Thus, a mixture as contemplated herein (II.) can be continuously applied to another Component (I.). A further layer of this other Component (I) can also subsequently be applied to the other side of the extrudate (II.), which is not yet covered with such a layer, so that a sandwich arrangement results, or another mixture can be applied as a third layer after an analogous manufacturing process. Another possibility is that a Mixture (II.) is added continuously or in portions to prefabricated moulds made of a further Component (I.) and then completely enveloped by an additional quantity of this further Component (I.), for example in the form of two hemispheres. The final density of the mixture can also be adjusted via the pressure.

Foamed solidified emulsions as such have already been mentioned above, but it should be noted here that the consistency of the foamed extrudate and the corresponding production facilities make it possible to create imaginative shapes, such as those familiar from meringue.

It is also possible to pour the solid cosmetic compositions as contemplated herein into a crucible, for example a glass jar. Since the strength of these compositions is in a range that allows an application portion to be taken manually, without a tool, from a crucible intended for multiple applications. If the composition has been foamed in a crucible during its manufacture, the result is a particularly interesting feeling.

It is also possible to make up solid cosmetic compositions reminiscent of a piece of paper, a foil or a wafer, which brings with it a new and pleasant feeling during application. Since the thickness of the coating is small in this type of packaging, short dissolution times are possible, which accommodates impatient users and does not encourage wasteful use of water. A product packaged in this way may be placed on the market in a packaging unit in which large number of leaves or flakes are placed in a small carton, possibly subdivided, so that a single withdrawal is possible.

After various designs and their respective advantages were explained in detail, the presentation of exemplary compositions and an exemplary manufacturing process follows.

Basic exemplary compositions of Component II. are shown in the following tables 1-4

TABLE 1

| Ingredient | Z1 | Z2 | Z3 | Z4 | Z5 | Z6 | Z7 | Z8 | Z9 |
|---|---|---|---|---|---|---|---|---|---|
| a): cationic surfactant | 0.1-15 | 0.1-14 | 0.15-12.5 | 0.2-11 | 0.25 10 | 0.25-9.5 | 0.3-9.0 | 0.4-8.5 | 0.5-7.5 |
| b): Polyvalent C₂-C₄ | 10-45 | 11-44 | 12.5-42.5 | 14-41 | 15-40 | 16-40 | 17.5-40 | 19-40 | 20.0-40 |
| alcohol | | | | | | | | | |
| c): saturated or | 1.0-15 | 1.25-14 | 1.5-13.5 | 1.75-13 | 2.0-12 | 3.0-12 | 5.0-12 | 6.0-12 | 8.0-12 |
| unsaturated, branched | | | | | | | | | |
| or unbranched C₈-C₃₀ | | | | | | | | | |
| alcohol, and/or | | | | | | | | | |
| saturated or unsaturated, | | | | | | | | | |
| branched or unbranched | | | | | | | | | |
| C₈-C₃₀ carboxylic acid | | | | | | | | | |
| and/or its salts | | | | | | | | | |
| d): Polysaccharide | 1.0-20 | 1.25-19 | 1.5-17.5 | 1.75-16 | 5.0-15 | 6.0-14 | 7.0-13.5 | 7.5-13 | 8.0-12 |
| Water and, if necessary, | ad | ad | ad | ad | ad | ad | ad | ad | ad |
| other auxiliary materials | 100 | 100 | 100 | 100 | 100 | 100 | 100 | 100 | 100 |
| and additives | | | | | | | | | |

TABLE 2

| Ingredient | Z11 | Z11 | Z12 | Z13 | Z14 | Z15 | Z16 | Z17 | Z18 |
|---|---|---|---|---|---|---|---|---|---|
| C₈-C₂₄ Alkyl | 0.1-15 | 0.1-14 | 0.15-12.5 | 0.2-11 | 0.25 10 | 0.25-9.5 | 0.3-9.0 | 0.4-8.5 | 0.5-7.5 |
| trimethylammonium | | | | | | | | | |
| salts | | | | | | | | | |
| 1.2-propylene glycol, | 10-45 | 11-44 | 12.5-42.5 | 14-41 | 15-40 | 16-40 | 17.5-40 | 19-40 | 20-40 |
| 1.3-butylene glycol, | | | | | | | | | |
| dipropylene glycol, | | | | | | | | | |
| glycerol and/or | | | | | | | | | |
| diglycerol | | | | | | | | | |
| Coco acids, lauric acid, | 1.0-15 | 1.25-14 | 1.5-13.5 | 1.75-13 | 2.0-12 | 3.0-12 | 5.0-12 | 6.0-12 | 8.0-12 |
| myristic acid, palmitic | | | | | | | | | |
| acid, stearic acid, | | | | | | | | | |
| behenic acid, oleic acid, | | | | | | | | | |
| coco alcohols, lauryl | | | | | | | | | |
| alcohol, myristyl | | | | | | | | | |
| alcohol, cetyl alcohol, | | | | | | | | | |
| stearyl alcohol, behenyl | | | | | | | | | |
| alcohol and/or oleyl | | | | | | | | | |
| alcohol | | | | | | | | | |
| Starch fractions from | 1.0-20 | 1.25-19 | 1.5-17.5 | 1.75-16 | 5.0-15 | 6.0-14 | 7.0-13.5 | 7.5-13 | 8.0-12 |
| corn, potatoes, rice, | | | | | | | | | |
| wheat and/or tapioca | | | | | | | | | |
| and/or | | | | | | | | | |
| Starch fractions from | | | | | | | | | |
| corn, potatoes, rice, | | | | | | | | | |
| wheat and/or tapioca | | | | | | | | | |
| and/or | | | | | | | | | |
| Derivatives of starches | | | | | | | | | |
| such as amylose, | | | | | | | | | |
| amylopectin, dextrins | | | | | | | | | |
| Water and, if necessary, | ad | ad | ad | ad | ad | ad | ad | ad | ad |
| other auxiliary | 100 | 100 | 100 | 100 | 100 | 100 | 100 | 100 | 100 |
| materials and additives | | | | | | | | | |

TABLE 3

| Ingredient | Z19 | Z20 | Z21 | Z22 | Z23 | Z24 | Z25 | Z26 | Z27 |
|---|---|---|---|---|---|---|---|---|---|
| Cetrimonium chloride | 0.1-15 | 0.1-14 | 0.15-12.5 | 0.2-11 | 0.25 10 | 0.25-9.5 | 0.3-9.0 | 0.4-8.5 | 0.5-7.5 |
| and/or Behentrimonium | | | | | | | | | |
| chloride | | | | | | | | | |
| Glycerine | 10-45 | 11-44 | 12.5-42.5 | 14-41 | 15-40 | 16-40 | 17.5-40 | 19-40 | 20-40 |
| Palmitic acid, stearic | 1.0-15 | 1.25-14 | 1.5-13.5 | 1.75-13 | 2.0-12 | 3.0-12 | 5.0-12 | 6.0-12 | 8.0-12 |
| acid, cetyl alcohol and/or | | | | | | | | | |
| stearyl alcohol | | | | | | | | | |
| Starch fractions from | 1.0-20 | 1.25-19 | 1.5-17.5 | 1.75-16 | 5.0-15 | 6.0-14 | 7.0-13.5 | 7.5-13 | 8.0-12 |
| corn and/or | | | | | | | | | |
| hydroxypropyl Starch | | | | | | | | | |
| phosphates and/or | | | | | | | | | |
| maltodextrin | | | | | | | | | |
| Water and, if necessary, | ad | ad | ad | ad | ad | ad | ad | ad | ad |
| other auxiliary materials | 100 | 100 | 100 | 100 | 100 | 100 | 100 | 100 | 100 |
| and additives | | | | | | | | | |

TABLE 4

| Ingredient | Z28 | Z29 | Z30 | Z31 | Z32 | Z33 | Z34 | Z35 | Z36 |
|---|---|---|---|---|---|---|---|---|---|
| a): cationic surfactant | 0.1-15 | 0.1-15 | 0.1-15 | 0.1-15 | 0.25 10 | 0.25 10 | 0.25 10 | 0.25 10 | 0.5-7.5 |
| b): Polyvalent C₂-C₄ | 10-45 | 10-45 | 10-45 | 10-45 | 15-40 | 15-40 | 15-40 | 15-40 | 20.0-40 |
| alcohol | | | | | | | | | |
| c): saturated or | 1.0-15 | 1.0-15 | 1.0-15 | 1.0-15 | 2.0-12 | 2.0-12 | 2.0-12 | 2.0-12 | 8.0-12 |
| unsaturated, branched or | | | | | | | | | |
| unbranched C₈-C₃₀ | | | | | | | | | |
| alcohol, and/or | | | | | | | | | |
| saturated or unsaturated, | | | | | | | | | |
| branched or unbranched | | | | | | | | | |
| C₈-C₃₀ carboxylic acid | | | | | | | | | |
| and/or its salts | | | | | | | | | |
| d): Polysaccharide | 1.0-20 | 1.0-20 | 1.0-20 | 1.0-20 | 5.0-15 | 5.0-15 | 5.0-15 | 5.0-15 | 8.0-12 |
| f): Oil, fat and/or wax | 0.01-10 | | | | 0.1-7 | | | | 0.5-5 |
| component | | | | | | | | | |
| Vegetable butter | | 0.01-10 | | 0.01-10 | | 0.1-7 | | 0.1-7 | |
| Triglyceride oil | | | 0.01-10 | 0.01-10 | | | 0.1-7 | 0.1-7 | |
| Water and, if necessary, | ad | ad | ad | ad | ad | ad | ad | ad | ad |
| other auxiliary materials | 100 | 100 | 100 | 100 | 100 | 100 | 100 | 100 | 100 |
| and additives | | | | | | | | | |

Detailed exemplary compositions of Component II. are shown in the following table Table 5

TABLE 5

| | | active ingredients | | | |
|---|---|---|---|---|---|
| Group | Ingredients | contained therein | Z37 | Z38 | Z39 |
| 1 | Water | Water | 18 | 36.5 | 36.5 |
| 1 | Citric acid | Citric acid | 0.25 | 0.25 | 0.25 |
| | monohydrate | | | | |
| 1 | Dehyquart A CA ® | Cetrimonium | 8 | 8 | 8 |
| | | chloride | | | |
| 1 | Glycerine 99.5% | Glycerine | 35 | 35 | 35 |
| 1 | Cetearyl alcohol | Cetearyl alcohol | 5 | 2 | 2 |
| 1 | Cutina FS 45 ® | Palmitic acid, | 5 | 2 | 2 |
| | | stearic acid | | | |
| 1 | Cutina GMS-V ® | Glyceryl stearate | 5 | 2.5 | 2.5 |
| 2 | Agenamalt ® 20.225 | Maltodextrin | 1 | 1 | 1 |
| | Maltodextrin DE15 | | | | |
| 3a | Structure XL ® | Hydroxypropyl | 1.5 | 1.5 | 1.5 |
| | (28-030A) | starch phosphate | | | |
| 3b | Maisita 9040 ® | Zea Mays- (Corn-) | 17.25 | 10 | 10 |
| | | starch | | | |
| 4 | Cetiol SB 45 ® | Butyrospermum Parkii | 0.5 | 0.5 | 0.5 |
| | | (Shea) Butter) | | | |
| 4 | Apricot kernel oil, | Prunus Armeniaca | 2 | 2 | 2 |
| | cold pressed | (apricot) seed oil | | | |
| 4 | Perfume Tea | Perfume (Scent) | 0.5 | 0.5 | 0.5 |
| | Grandiosa 611084 | | | | |
| 4 | Phenoxyethanol, | Phenoxyethanol | 1 | 1 | 1 |
| | pure | | | | |

The exemplary process for preparing compositions 37 to 39 was carried out as follows:

The ingredients were used in the ratio shown in the table above.

Dehyquart A CA was heated in a drum to about 40° C. to 50° C. and mixed in case of an uneven distribution of its ingredients. After mixing until homogeneity, the other ingredients of group 1 (see table 2) were added. It was mixed again until homogeneity and then the temperature was increased to about 85° C. to 90° C. At this temperature, the ingredients of Group 2 (see Table 2) were added and mixed in until homogeneous. This was then repeated with the ingredients of Group 3 (3a and 3b, see Table 2). The ingredients of Group 4 (see Table 2) were homogeneously mixed together and also added to the previously prepared mixture and mixed in until homogeneity was achieved. After that, the temperature was no longer actively maintained at about 85° C. to 90° C., but it was only ensured that it did not drop to 70° C. or less. Finally, the mixture was kept at a temperature above 70° C. for filling or packaging.

This was followed by the introduction of a gas chosen from air, N₂, N₂O and/or CO₂ at a pressure of about 2 to 40 bar or alternatively the introduction of air with a high speed mixer, extrusion of the gassed mixture from a desired shaping orifice or into a desired mould and solidification/cooling of the extrudate in the desired shape and removal of the extrudate from the mould or cutting and portioning of the extrudate. The cosmetic compositions obtained had densities ranging from about 0.2 g/cm³ to 1.2 g/cm³.

Up to this point, the process product corresponded to a previously described Component II. and it was further temporarily stored as a separate layer between two layers of a Component I. (comprising circular wafers of >about 90 wt. % potato starch and <about 10 wt. % water; based on the total weight of Component I.) (Sandwich arrangement) to obtain a cosmetic composition as contemplated herein.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A solid cosmetic composition comprising two separate Components I. and II., wherein
the at least one Component I. comprises at least one of:
a. starch fractions from corn, potatoes, rice, wheat and/or tapioca,
b. cereal flour, and/or
c. sugar selected from the group including glucose, sucrose, fructose, maltose, and
the at least one Component II. comprises:
a. from about 0.1 to 15.0% by weight of at least one cationic surfactant,
b. from about 10.0 to 45.0% by weight of at least one polyhydric C₂-C₆ alcohol,
c. from about 1.0 to 15.0% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
d. from about 1.0 to 20.0% by weight of at least one starch fraction, a modified starch and/or a starch derivative,
where the quantities stated refer to the total weight of Component II.

2. The solid cosmetic composition according to claim 1, comprising in Component I.—based on its total weight—from about 75 to 100% by weight of one or more ingredient(s) from groups a. to c.

3. The solid cosmetic composition according to claim 1, comprising C₈-C₃₀ alkyl tri-C₁-C₄ alkylammonium salts.

4. The solid cosmetic composition according to claim 1, comprising cationic surfactant salts known under the INCI designation “Cetrimonium” and/or “Behentrimonium”.

5. The solid cosmetic composition according to claim 1, comprising as polyhydric alcohol b) in Component II. 1,2-propylene glycol, 1,3-butylene glycol, dipropylene glycol, glycerol and/or diglycerol.

6. The solid cosmetic composition according to claim 2, comprising as polyhydric alcohol b) in Component II. 1,2-propylene glycol, 1,3-butylene glycol, dipropylene glycol, glycerol and/or diglycerol.

7. The solid cosmetic composition according to claim 3, comprising as polyhydric alcohol b) in Component II. 1,2-propylene glycol, 1,3-butylene glycol, dipropylene glycol, glycerol and/or diglycerol.

8. The solid cosmetic composition according to claim 4, comprising as polyhydric alcohol b) in Component II. 1,2-propylene glycol, 1,3-butylene glycol, dipropylene glycol, glycerol and/or diglycerol.

9. The solid cosmetic composition according to claim 1, comprising as Component c) in Component II. saturated or unsaturated, branched or unbranched C₈-C₃₀ carboxylic acids and/or saturated or unsaturated, branched or unbranched C₈-C₃₀ alcohols.

10. The solid cosmetic composition according to claim 1, comprising as Component c) in Component II. C₁₀-C₂₂ carboxylic acids and/or C₁₀-C₂₂ alcohols.

11. The solid cosmetic composition according to claim 1, comprising as Component c) in Component II. coco acids, Lauric acid, myristic acid, palmitic acid, stearic acid, behenic acid, oleic acid and mixtures thereof and/or coconut alcohols, lauryl alcohol, myristyl alcohol, cetyl alcohol, stearyl alcohol, behenyl alcohol, oleyl alcohol and mixtures thereof.

12. The solid cosmetic composition according to claim 2, comprising as Component c) in Component II. saturated or unsaturated, branched or unbranched C₈-C₃₀ carboxylic acids and/or saturated or unsaturated, branched or unbranched C₈-C₃₀ alcohols.

13. The solid cosmetic composition according to claim 2, comprising as Component c) in Component II. C₁₀-C₂₂ carboxylic acids and/or C₁₀-C₂₂ alcohols.

14. The solid cosmetic composition according to claim 2, comprising as Component c) in Component II. coco acids, Lauric acid, myristic acid, palmitic acid, stearic acid, behenic acid, oleic acid and mixtures thereof and/or coconut alcohols, lauryl alcohol, myristyl alcohol, cetyl alcohol, stearyl alcohol, behenyl alcohol, oleyl alcohol and mixtures thereof.

15. The solid cosmetic composition according to claim 1, comprising as polysaccharide d) in Component II.
i. Starch fractions from corn, potatoes, rice, wheat and/or tapioca and/or
ii. Modified starches from corn, potatoes, rice, wheat and/or tapioca and/or
iii. Derivatives of starches such as amylose, amylopectin, dextrins.

16. The solid cosmetic composition according to claim 15, wherein the polysaccharides d) comprise starch fractions from maize, compounds known under the INCI designation Hydroxypropyl Starch Phosphates and maltodextrin.

17. The solid cosmetic composition according to claim 1,
comprising in Component I.—based on its total weight—from about 75 to 100% by weight of one or more ingredient(s) from groups a. to c.,
comprising cationic surfactant salts known under the INCI designation “Cetrimonium” and/or “Behentrimonium”,
comprising glycerol as polyhydric alcohol b) in Component II,
comprising as Component c) in Component II. coco acids, Lauric acid, myristic acid, palmitic acid, stearic acid, behenic acid, oleic acid and mixtures thereof and/or coconut alcohols, lauryl alcohol, myristyl alcohol, cetyl alcohol, stearyl alcohol, behenyl alcohol, oleyl alcohol and mixtures thereof, and
comprising as polysaccharide d) in Component II. starch fractions chosen from maize, compounds known under the INCI designation Hydroxypropyl Starch Phosphates and maltodextrin.

18. The solid cosmetic composition according to claim 1, wherein
Component I. is coated with Component II., or
Component I. is impregnated, sprayed or coated with Component II., or
Component II. Is rolled onto Component I., or
Component I. with Component II. is printed.

19. The solid cosmetic composition according to claim 1,
where Component II. is disposed as a separate layer and sandwiched between two Components I. or
comprising an additional layer III, which comprises a hair cleansing agent or
where Component II. is completely enveloped by Component I. or
Component II. is in the form of a porous body which has a density of from about 0.2 g/cm³ to 1.2 g/cm³ and which converts into an emulsion when in contact with water.

20. A cosmetic method for hair care wherein the solid cosmetic composition according to claim 1 is moistened with water, soaked and/or emulsified, rubbed between the hands and distributed on the hair, and after an exposure time of from about 5 seconds to 5 minutes is optionally rinsed out again with water.
